(19) United States
(12) Patent Application Publication
(10) Pub. No.: US 2003/0069202 A1
(43) Pub. Date: Apr. 10, 2003

(54) Compositions, kits, and methods for promoting defined health benefits

(76) Inventors: Kenneth Norman Kern, Cincinnati, OH (US); Matthew Thomas Heisey, Wyoming, OH (US)

Correspondence Address: THE PROCTER & GAMBLE COMPANY, PATENT DIVISION, IVORYDALE TECHNICAL CENTER - BOX 474, 5299 SPRING GROVE AVENUE, CINCINNATI, OH, 45217, US

(21) Appl. No.: 09/760,280

(22) Filed: Jan. 12, 2001

Related U.S. Application Data
(63) Continuation-in-part of application No. 09/586,213, filed on Jun. 2, 2000, now abandoned.

(51) Int. Cl. A61K 31/728; A61K 31/727; A61K 31/726; A61K 31/7076; A61K 31/70

(52) U.S. Cl. 514/46; 514/54; 514/56; 514/62

(57) ABSTRACT

The present invention is directed to compositions comprising:

(a) a first component selected from the group consisting of gelatin, cartilage, aminosugars, glycosaminoglycans, methylsulfonylmethane, precursors of methylsulfonylmethane, S-adenosylmethionine, salts thereof, and mixtures thereof; and

(b) a second component comprising:

(i) a cation source selected from the group consisting of calcium, potassium, magnesium, and mixtures thereof; and

(ii) an edible acid source.

The present invention is further directed to food, beverage, pharmaceutical, over-the- counter, and dietary supplement products, which comprise the present compositions. The invention also relates to kits comprising the present compositions and information that use of the composition promotes one or more of the presently defined health benefits, including joint health, bone health, cardiac health, and anti-inflammation. The present invention additionally relates to methods of treating joint function, bone function, cardiac function, or inflammation comprising administering to a mammal a composition as defined herein.

### REFERENCE TO PRIORITY APPLICATIONS

The present application claims priority to co-pending U.S. Application Serial No. 09/586,213 filed Jun. 2, 2000.

### FIELD OF THE INVENTION

The present invention is directed to compositions which are useful for promoting one or more health benefits including, for example, joint health, bone health, cardiac health, and/or anti-inflammation. The present invention is further directed to kits comprising the compositions and methods of using the compositions and kits.

### BACKGROUND OF THE INVENTION

Osteoarthritis is a widespread, degenerative disease of the joints, cartilage, and other articular components. Osteoarthritis affects all ethnic groups worldwide. In addition to humans, osteoarthritis affects nearly all mammals, for example, horses and cows, as well as domestic cats and dogs. Many treatments for osteoarthritis have been proposed, all resulting in varying degrees of success.

One osteoarthritis treatment which has been recently proposed is oral administration of chondroprotective agents such as glucosamine and/or chondroitin. See e.g., Henderson, U.S. Pat. No. 5,364,845, assigned to Nutramax Laboratories, issued Nov. 15, 1994. Indeed, various commercial products are available in the marketplace, including nutritional supplements containing such agents and powders which may be formulated into beverage compositions immediately prior to use.

Typically, administration of such agents is designed to enhance proteoglycan through an increased concentration of glycosaminoglycans. Enhanced proteoglycan provides the framework for collagen and other joint components, as well as imparting flexibility, resiliency, and resistance to compression. Thus, these agents may be administered according to various methods to enhance the articular compositions or, at a minimum, inhibit the process of degradation.

However, readily available compositions are not designed to combat all factors leading to the degradation of joints and bones. Indeed, while the foregoing examples glucosamine and/or chondroitin are useful, these compounds are not, by themselves, useful for treating all aspects associated with the osteoarthritic state. Thus, it will be important to discover new compositions which more broadly meet the needs of the osteoarthritic, or pre-osteoarthritic (e.g., athletic) subject.

The present inventors have discovered that administration of a mineral, e.g., calcium, with a component designed to enhance joint function is critically important in the furtherance of joint health in the osteoarthritic, or pre-osteoarthritic subject. Inclusion of this mineral has been found to be particularly important, since those subjects affected by osteoarthritis will typically need increased bioavailable calcium relative to non-arthritic subjects. Even further, the present inventors have discovered that the particular mineral form utilized, i.e., the form specifically defined herein, is critical for this purpose. Indeed, the specific mineral form utilized herein provides enhanced bioavailability relative to other sources of minerals, and thus acts synergistically with the joint health component.

Additionally, as previously stated, oral administration of compounds such as glucosamine and/or chondroitin has been a prominent commercially available therapy. Various commercial products are available in the marketplace, including powders which may be formulated into beverage compositions immediately prior to use. Indeed, immediate formulation has historically been found important for preservation of stability, since it is well-known that glucosamine, and other similar components, exhibit instability in the presence of aqueous solutions or even merely in the presence of hydrated atmosphere. The present inventors have surprisingly discovered that addition of an acidic second component to the chondroprotective agent, i.e., the defined mineral form, maintains the stability of such agent. Indeed, the present inventors have discovered that the presently described acidic compatible matrix optimizes the stability of the chondroprotective agent, e.g., glucosamine. This is in contrast to inclusion of more basic components, e.g., a carbonate, which liberates basic carbon dioxide. Thus, in view of this discovery, more efficacious compositions containing a chondroprotective agent are provided. Additionally, stable, ready-to-drink beverage compositions can now be made available to consumers through this invention. These ready-to-drink embodiments will improve consumer acceptability and compliance, resulting in improved health of the consumer.

Accordingly, the present inventors describe herein compositions which comprise a component designed to enhance joint function together (i.e., a chondroprotective agent) with the specifically defined acidic mineral source. Kits comprising these compositions, as well as methods of their use are also described.

### SUMMARY OF THE INVENTION

The present invention is directed to compositions which are useful for promoting one or more health benefits as defined herein. In particular, the present invention is directed to compositions comprising:

(a) a first component selected from the group consisting of gelatin, cartilage, aminosugars, glycosaminoglycans, methylsulfonylmethane, precursors of methylsulfonylmethane, S-adenosylmethionine, salts thereof, and mixtures thereof; and

(b) a second component comprising:

(i) a cation source selected from the group consisting of calcium, potassium, magnesium, and mixtures thereof; and

(ii) an edible acid source.

The present invention is further directed to food, beverage, pharmaceutical, over-the-counter, and dietary supplement products which comprise the present compositions. The products are suitable for mammalian use. The invention also relates to kits comprising the present compositions and information that use of the composition promotes one or more of the presently defined health benefits, including joint health, bone health, cardiac health, and anti- inflammation. The present invention additionally relates to methods of treating joint function, bone function, cardiac function, or inflammation comprising administering to a mammal a composition as defined herein.

### DETAILED DESCRIPTION OF THE INVENTION

The present invention is directed to compositions which are useful, for example, in food, beverage, pharmaceutical, over-the-counter, and dietary supplement products. The food and beverage products include those which are traditional, as well as those which may be classified as, for example, “medical foods” or “dietary supplements” under regulatory guidelines. The compositions are suitable for mammalian use, particularly use in humans and domestic animals such as, for example, dogs, cats, horses, and cows. The present invention is further directed to kits comprising such compositions and methods of using such compositions.

The compositions of the present invention are useful for providing one or more joint health, bone health, cardiac health, and/or anti-inflammation benefits. Joint health benefits include, but are not limited to, preventing, inhibiting, ceasing and/or reversing the actions associated with arthritis, particularly osteoarthritis. Thus, improved joint health will provide, for example, decreased pain in the joints and/or increased flexibility. Bone health benefits include, but are not limited to, preventing, inhibiting, ceasing, and/or reversing bone loss and/or building bone mass, and/or preventing, inhibiting, ceasing, and/or reversing osteoporosis. Thus, improved bone health may provide, for example, healthy bones, stronger bones, and/or increased bone mass. Cardiac health benefits include, but are not limited to, preventing, inhibiting, ceasing, and/or reversing, for example, heart disease, atherosclerosis, and/or restenosis. Anti-inflammation benefits include, for example, preventing, inhibiting, ceasing, and/or reversing inflammation, particularly in the joints. Thus, anti-inflammation will typically result in pain reduction.

Publications and patents are referred to throughout this disclosure. All references cited herein are hereby incorporated by reference.

All percentages and ratios are calculated by weight unless otherwise indicated. All percentages and ratios are calculated based on the total composition unless otherwise indicated.

All component or composition levels are in reference to the active level of that component or composition, and are exclusive of impurities, for example, residual solvents or by-products, which may be present in commercially available sources.

Referred to herein are trade names for components including various ingredients utilized in the present invention. The inventors herein do not intend to be limited by materials under a certain trade name. Equivalent materials (e.g., those obtained from a different source under a different name or catalog (reference) number) to those referenced by trade name may be substituted and utilized in the compositions, kits, and methods herein.

In the description of the invention various embodiments and/or individual features are disclosed. As will be apparent to the ordinarily skilled practitioner, all combinations of such embodiments and features are possible and can result in preferred executions of the present invention.

The compositions, kits, and methods herein may comprise, consist essentially of, or consist of any of the elements as described herein.

### Composition of the Present Invention

The present invention is directed to compositions which are useful in, for example, food, beverage, pharmaceutical, over-the-counter, and dietary supplement products. The products are suitable for mammalian use, particularly use in humans and domestic animals such as, for example, dogs, cats, horses, and cows. Preferably, the present compositions are directed for use in humans and domestic animals. More preferably, the present compositions are directed for use in humans, domestic dogs, and domestic cats. Most preferably, the present compositions are directed for use in humans.

Formulation of chondroprotective powder compositions in water immediately prior to consumption has historically been found important for preservation of stability, since it is well-known that glucosamine, and other similar components, exhibit instability in the presence of aqueous solutions or even merely in the presence of hydrated atmosphere. The present inventors have surprisingly discovered that addition of an acidic second component to the chondroprotective agent, i.e., the defined mineral form, maintains the stability of such agent. Indeed, the present inventors have discovered that the presently described acidic compatible matrix optimizes the stability of the chondroprotective agent, e.g., glucosamine. This is in contrast to inclusion of more basic components only, e.g., a carbonate without addition of an edible acid source (which liberates basic carbon dioxide).

Consistent with the present discovery, the compositions of this invention preferably exhibit a pH of from about 2 to about 8, more preferably from about 2 to about 8, still more preferably from about 2 to about 5, even more preferably from about 3 to about 5, and most preferably from about 3.5 to about 4.5.

In accordance with the present invention, therefore, the present compositions comprise:

(a) a first component selected from the group consisting of gelatin, cartilage, aminosugars, glycosaminoglycans, methylsulfonylmethane, precursors of methylsulfonylmethane, S-adenosylmethionine, salts thereof, and mixtures thereof; and

(b) a second component comprising:

(i) a cation source comprising an element selected from the group consisting of calcium, potassium, magnesium, and mixtures thereof; and

(ii) an edible acid source.

The various components of the present compositions will be described further herein. The compositions are particularly suited for treating joint function, bone function, cardiac function, or inflammation in a mammal. More preferably, the compositions are useful for treating joint function, bone function, and inflammation in a mammal. Most preferably, the compositions are useful for treating joint function and inflammation in a mammal, particularly joint function.

The First Component

The first component of the present compositions is a chondroprotective agent selected from the group consisting of gelatin, cartilage, aminosugars, glycosaminoglycans, methylsulfonylmethane, precursors of methylsulfonylmethane, S-adenosylmethionine, salts thereof, and mixtures thereof. This component is a key component particularly useful for promoting bone, joint, and cardiac health, most preferably bone and joint health. The term “first component” is utilized herein for convenience and should not be construed as, for example, indicating relative importance or an order of administration.

The first component, which is selected from gelatin, cartilage, aminosugars, glycosaminoglycans, methylsulfonylmethane, precursors of methylsulfonylmethane, S-adenosylmethionine, as well as their salts and mixtures, are particularly useful for bone and joint function, particularly joint function. Without intending to be limited by theory, the first component is important for enhancing joint function as the component aids in the stimulation of proteoglycan and collagen in vivo. Proteoglycan provides the connective tissue, for example, collagen, which is necessary for joint health. Indeed, proteoglycan is comprised of glycosaminoglycans (often termed “GAGs”) which are long chains of modified sugars. Aminosugars and methylsulfonylmethane are useful for building glycosaminoglycans and proteoglycan. Additionally, the cardiac benefits of various of these components is also a beneficial feature of this component. See e.g., Morrison et al., Coronary Heart Disease and the Mucopolysaccharides (Glycosaminoglycans), pp. 109-127 (1973).

Preferably, the first component is selected from gelatin, cartilage, aminosugars, glycosaminoglycans, S-adenosylmethionine, salts thereof, and mixtures thereof. More preferably, the first component is selected from aminosugars, glycosaminoglycans, S-adenosylmethionine, salts thereof, and mixtures thereof. Even more preferably, the first component is selected from aminosugars, glycosaminoglycans, salts thereof, and mixtures thereof. Most preferably, the first component is a salt of an aminosugar, particularly wherein the aminosugar is glucosamine.

The various first components, and preferred embodiments thereof, are described in expanded detail as follows. With respect to dosing preferences, all dosage levels are based on typical human subjects (e.g., about a 55 to 65 kg subject). Wherein the present composition is used in other mammals, it may be necessary to modify the dosage. Modification of dosages based on the needs of the subject is well within the skill of the ordinary artisan. It therefore understood that these dosage ranges are by way of example only, and that daily administration can be adjusted depending on various factors. The specific dosage of the chondroprotective agent to be administered, as well as the duration of treatment are interdependent. The dosage and treatment regimen will also depend upon such factors as the specific chondroprotective agent used, the treatment indication, the efficacy of the compound, the personal attributes of the subject (such as, for example, weight, age, sex, and medical condition of the subject), and compliance with the treatment regimen.

Gelatin

As is commonly known, gelatin is a protein obtained from the partial hydrolysis of collagen, which is the major structural and connective protein tissue in mammals. Gelatin typically contains from about 84% to about 90% protein, from about 1% to about 2% mineral salts, and from about 8% to about 15% water (these are non-limiting approximations). Gelatin typically contains specific amounts of 18 different amino acids, which are joined together to form polypeptide chains of approximately 1,000 amino acid residues per chain.

Typically, the collagen obtained for gelatin production is from animal bones and skins, e.g., from cows and pigs. Gelatin production will typically involve the subjection of collagenous material to alkaline pre-treatment, followed by hot-water extraction (providing gelatin having an iso-electric point of about 5). Acidic pre-treatment may also be utilized (providing gelatin having an iso-electric point of from about 7 to 9).

In accordance with the present invention, wherein gelatin is included within a present composition, a single dose of gelatin within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising gelatin is dosed from about once to about five times daily. However, in the food and beverage composition embodiments of the present invention, which are preferred, a typical dosage can be increased accordingly such that dosing need only occur about once daily. Thus, in these food and beverage compositions, compliance and consumer benefit is enhanced.

Cartilage

Cartilage may be chosen as the first component in the present compositions. As is commonly known in the art, cartilage is a tough, elastic tissue present in the joints (as well as other locations) of the bodies of various mammals. Cartilage is comprised of at least one of calcium, proteins, carbohydate mucopolysaccharides (e.g., chondroitin), and collagen.

Particularly preferred for use herein is bovine cartilage and shark cartilage. Bovine cartilage is primarily derived from the trachea of cows (also known as bovine tracheal cartilage, or BTC). It is similar in structure to shark cartilage. Shark cartilage is a widely utilized cartilage source, as the skeletons of sharks are primarily composed of cartilage rather than bone.

In accordance with the present invention, wherein cartilage is included within a present composition, a single dose of cartilage within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising cartilage is dosed from about once to about five times daily. However, in the food and beverage composition embodiments of the present invention, which are preferred, a typical dosage can be increased accordingly such that dosing need only occur about once daily. Thus, in these food and beverage compositions, compliance and consumer benefit is enhanced.

Aminosugars

One or more aminosugars may be chosen as the first component herein. The aminosugars are monosaccharide components (ie., hexoses) which are modified with an amine functionality. The amine functionality may be a free amine moiety or a protected amine moiety (e.g., N-acetyl amine). Preferably, the aminosugar is a precursor to glycosaminoglycan, which is important for construction of joint constituents (e.g., collagen). Additionally, certain aminosugars may serve to inhibit the activity of enzymes which are implicated in breakdown the cartilage in osteoarthritics (e.g., mannosamine, which has been discovered to inhibit aggrecanase). The aminosugars are well-known in the art; many aminosugars are naturally occurring.

Particularly preferred aminosugars include glucosamine, salts of glucosamine, galactosamine, salts of galactosamine, mannosamine, salts of mannosamine, as well as the N-acetyl derivatives of the foregoing, including N-acetyl glucosamine and N-acetyl galactosamine. More preferably, the aminosugars include glucosamine and salts of glucosamine, most preferably salts of glucosamine. Particularly preferred salts of glucosamine include glucosamine sulfate and glucosamine hydrochloride. The salts of glucosamine are particularly preferred to aid bioavailability of the aminosugar in addition to the bioavailability benefit achieved by the second component (as described herein below).

As an example, glucosamine provides the building block needed in vivo to manufacture glycosaminoglycan, which is found in cartilage. Thus, glucosamine, and other aminosugars, function not only to relieve symptoms of joint pain but also stop, inhibit, and/or reverse the degenerative process.

Typical single dosing of the aminosugars is preferably from about 1 mg to about 5000 mg, more preferably from about 100 mg to about 3600 mg, even more preferably from about 150 mg to about 2200 mg, and most preferably from about 250 mg to about 1900 mg, based on the molecular weight of glucosamine hydrochloride. For example, a particularly preferred dosage of glucosamine hydrochloride is about 1800 mg, which translates to about 1480 mg of glucosamine. All other aminosugars may be similarly dosed, based on the molecular weight of glucosamine hydrochloride. Typically, the composition comprising the aminosugar is dosed from about once to about five times daily, preferably from about once to about three times daily. However, in the food and beverage composition embodiments of the present invention, which are preferred, a typical dosage can be increased accordingly such that dosing need only occur about once daily.

Glycosaminoglycans

One or more glycosaminoglycans may be utilized as the first component herein. The glycosaminoglycans are commonly known as GAGs, and are precursors to joint structure, for example, proteoglycan. The glycosaminoglycans may also be important for the healing of bone.

Suitable glycosaminoglycans will be well-known to the ordinarily skilled artisan. Preferred glycosaminoglycans include chondroitin, hyaluronic acid, keratan, heparin, and dermatin, as well as salts of the foregoing. For example, chondroitin sulfate is a particularly preferred chondroitin salt. As with the aminosugars, salts of the glycosaminoglycans are particularly preferred for use herein.

As an example, chondroitin provides the structure and allows various molecules to transport through cartilage (which is important, since there is no blood supply to cartilage). Chondroitin is a major constituent of cartilage and contains repeating chains of saccharides.

Typical single dosing of the glycosaminoglycans is preferably from about 1 mg to about 10 grams, more preferably from about 100 mg to about 5 grams, even more preferably from about 150 mg to about 1000 mg, and most preferably from about 250 mg to about 800 mg, based on the molecular weight of chondroitin. All other glycosaminoglycans may be similarly dosed, based on the molecular weight of chondroitin. Typically, the composition comprising the glycosaminoglycan is dosed from about once to about five times daily. However, in the food and beverage composition embodiments of the present invention, which are preferred, a typical dosage can be increased accordingly such that dosing need only occur about once daily.

Methylsulfonylmethane and Precursors of Methylsulfonylmethane

The first component herein may also be methylsufonylmethane, or a precursor thereof. As used herein, the term “precursor thereof” means a compound which, in mammalian systems, is converted to methylsulfonylmethane in vivo. Methylsulfonylmethane, and precursors thereof, are common ingredients found in vivo and in nature, e.g., in unprocessed foods. Without intending to be limited by theory, it is believed that the sulfur moiety present in methylsulfonylmethane, and its precursors, provides the disulfide bridging (also commonly known as “tie-bars” or “cross-links”) necessary to hold the connective tissue in joints together.

While unprocessed foods contain methylsulfonylmethane, and the precursors thereof, conventional food processing and preparation causes the loss of these compounds from the foods. Therefore, commonly ingested foods may become deficient in these compounds. In these respects, methylsulfonylmethane is similar to vitamins and minerals which are typically partially or totally lost during normal food processing and preparation. It is therefore an important embodiment of this invention to include, as the first component, methylsulfonylmethane or a precursor thereof in the present compositions.

Non-limiting examples of precursors of methylsulfonylmethane include methionine and methyl sulfide. See e.g., Herschler et al., U.S. Pat. No. 4,863,748, issued Sept. 5, 1989. Precursors of methylsulfonylmethane is associated with a variety of health benefits, including joint benefits (such as relief from osteoarthritis and rheumatoid arthritis), as well as anti-inflammation.

In accordance with the present invention, wherein methanesulfonylmethane is included within a present composition, a single dose of methanesulfonylmethane within the composition is preferably from about 0.01 mg to about 2000 mg, more preferably from about 0.01 mg to about 500 mg, even more preferably from about 1 mg to about 200 mg, and most preferably from about 1 mg to about 100 mg. The precursors of methanesulfonylmethane may be similarly dosed, based on the molecular weights of the precursors relative to methanesulfonylmethane. Typically, the composition comprising methanesulfonylmethane is dosed from about once to about five times daily. However, in the food and beverage composition embodiments of the present invention, which are preferred, a typical dosage can be increased accordingly such that dosing need only occur about once daily.

S-Adenosylmethionine

S-adenosylmethionine, which is commonly known as SAM-e, is a compound which is found in most, if not all, living cells. Without intending to be limited by theory, SAM-e is produced through reaction of the essential amino acid methionine and the energy molecule known as adenosine triphosphate (commonly known as ATP). SAM-e manufactures the components of cartilage and repairs, restores, and maintains joint function. SAM-e is made in vivo from the amino acid methionine, and is found in ordinary dietary sources such as meats, soybeans, eggs, seeds, and lentils.

In accordance with the present invention, wherein SAM-e is included within a present composition, a single dose of SAM-e within the composition is preferably from about 1 mg to about 2000 mg, more preferably from about 100 mg to about 700 mg, even more preferably from about 150 mg to about 600 mg, and most preferably from about 200 mg to about 400 mg. Typically, the composition comprising SAM-e is dosed from about once to about five times daily. However, in the food and beverage composition embodiments of the present invention, which are preferred, a typical dosage can be increased accordingly such that dosing need only occur about once daily.

The Second Component

The second component utilized in the composition is a critical component which synergistically interacts with the first component to provide the health benefits described herein, particularly promotion of joint and/or bone health. In addition, it has been surprisingly discovered that the second component, as specifically defined herein, provides an acidic matrix in the composition which, unexpectedly, maintains the stability of the first component, particularly in aqueous solution. The term “second component” is utilized herein for convenience and should not be construed as, for example, indicating relative importance or an order of administration. In fact, as discussed, it has been discovered that both the first and second components of the present invention are needed for achieving the benefits herein.

The second component comprises:

(i) a cation source comprising an element selected from the group consisting of calcium, potassium, magnesium, and mixtures thereof; and

(ii) an edible acid source.

Reaction of the cation source and the edible acid source in situ, particularly in aqueous solution, provides a particularly preferred, highly soluble, and bioavailable second component. Thus, while the present disclosure describes these sources as separate components, it should be understood, and is intended by the inventors, that this separate description additionally includes in particular, by definition, their reactive cation-containing products. For example, the most preferred second component for use herein is calcium citrate malate (e.g., from Jost Chemicals, St. Louis, Mo.).

The second component utilized herein is described in, e.g., Mehansho et aL, U.S. Pat. No. 5,670,344, issued Sept. 23, 1997; Diehl et al., U.S. Pat. No. 5,612,026, issued Mar. 18, 1997; Andon et al., U.S. Pat. No. 5,571,441, issued Nov. 5, 1996; Meyer et al., U.S. Pat. No. 5,474,793, issued Dec. 12, 1995; Andon et al., U.S. Pat. No. 5,468,506, issued Nov. 21, 1995; Burkes et al., U.S. Pat. No. 5,445,837, issued Aug. 29, 1995; Dake et al., U.S. Pat. No. 5,424,082, issued Jun. 13, 1995; Burkes et al., U.S. Pat. No. 5,422,128, issued Jun. 6, 1995; Burkes et al., U.S. Pat. No. 5,401,524, issued Mar. 28, 1995; Zuniga et al., U.S. Pat. No. 5,389,387, issued Feb. 14, 1995; Jacobs, U.S. Pat. No. 5,314,919, issued May 24, 1994; Saltman et al., U.S. Pat. No. 5,232,709, issued Aug. 3, 1993; Camden et al., U.S. Pat. No. 5,225,221, issued Jul. 6, 1993; Fox et al., U.S. Pat. No. 5,215,769, issued Jun. 1, 1993; Fox et al, U.S. Pat. No. 5,186,965, issued Feb. 16, 1993; Saltman et al., U.S. Pat. No. 5,151,274, issued Sept. 29, 1992; Kochanowski, U.S. Pat. No. 5,128,374, issued Jul. 7, 1992; Mehansho et al., U.S. Pat. No. 5,118,513, issued Jun. 2, 1992; Andon et al., U.S. Pat. No. 5,108,761, issued Apr. 28, 1992; Mehansho et al., U.S. Pat. No. 4,994,283, issued Feb. 19, 1991; Nakel et al, U.S. Pat. No. 4,786,510, issued Nov. 22, 1988; and Nakel et al., U.S. Pat. No. 4,737,375, issued Apr. 12, 1988.

Preferred compositions of the present invention will comprise from about 0.0001% to about 3%, more preferably from about 0.01% to about 2.5%, even more preferably from about 0.03% to about 2%, and most preferably from about 0.05% to about 1% of the second component, by weight of the composition.

The Cation Source

The cation source is part of the second component of the present compositions. The cation source comprises an element selected from calcium, potassium, and magnesium, as well as mixtures thereof. Preferably, the cation source comprises an element selected from calcium, magnesium, and mixtures thereof. Most preferably, the cation source comprises calcium.

The cation source may be present, for example, as the respective carbonates, bicarbonates, hydrogen phosphates, dihydrogen phosphates, hydroxides, oxides, or sour salts, e.g., citrates or malates. The carbonates and hydroxides are particularly preferred herein, primarily for reasons related to favorable taste and enhanced bioavailability.

The Edible Acid Source

The edible acid source is critical to the present invention because it aids in solubilizing the cation source which, in turn, enhances the bioavailability of the cation source as well as the first component herein. Preferably, the edible acid source is selected from lactic acid, citric acid, malic acid, fumaric acid, adipic acid, phosphoric acid, gluconic acid, tartaric acid, ascorbic acid, acetic acid, phosphoric acid, succinic acid, and mixtures thereof. More preferably, the edible acid source is selected from citric acid, malic acid, tartartic acid, fumaric acid, succinic acid, and mixtures thereof. Even more preferably, the edible acid source is selected from citric acid, malic acid, and mixtures thereof. Most preferably, the edible acid source is a mixture of citric acid and malic acid. Thus, for example, wherein the second component is calcium citrate malate (e.g., from Jost Chemicals, St. Louis, Mo.), the edible acid source is a mixture of citric acid and malic acid.

Wherein the edible acid source is a mixture of acids, the mixture is preferably of two acids. Any ratio of the various acids to the other respective acids may be utilized. However, wherein the mixture is of two acids (e.g., a first acid and a second acid), it is preferable that the ratio of first acid to second acid, by weight, is from about 5:95 to about 95:5, more preferably from about 20:80 to about 80:20, and most preferably from about 40:60 to about 60:40.

Optional Components of the Present Compositions

The compositions of the present invention may be utilized in food, beverage, pharmaceutical, and over-the-counter compositions. The food and beverage compositions may be available as, for example, typically marketed foods or beverages, or as dietary supplements or medical foods. Particularly preferred for use herein include syrups and concentrates suitable for dilution to provide a ready-to-drink beverage composition, powders or other dry compositions suitable for dilution to provide a ready-to-drink beverage composition, and ready-to-drink beverage compositions. Among these, ready-to-drink beverage compositions are most preferred, with dry compositions and concentrates also preferred. Preferred beverage compositions include fruit juices, coffees, teas, milks, and the like.

Food compositions are also useful herein. Preferred food compositions include chews, candies, gum, and other confectionery products, bars (including "health" bars and dessert bars), as well as other baked goods and spreads.

Tablets, capsules, pills, and other such forms are also useful herein.

Consistent with these various uses, the compositions of the present invention may comprise additional optional components to enhance, for example, their performance in providing joint health, bone health, other health benefits, a desirable nutritional profile, and/or organoleptic properties. For example, one or more omega-3-fatty acids, bracers, flavanols, milk solids, soluble fibers, non-caloric sweeteners, nutrients, flavoring agents, coloring agents, preservatives, acidulants, emulsifiers, thickeners, oils, water, carbonation components, and the like may be included in the compositions herein. Such optional components may be dispersed, solubilized, or otherwise mixed into the present compositions. These components may be added to the compositions herein provided they do not substantially hinder the properties of the composition, particularly the provision of joint and/or bone health. Non-limiting examples of optional components suitable for use herein are given below.

Omega-3 -Fatty Acids

In a particularly preferred embodiment of the present invention, one or more omega-3-fatty acids may be added to the present compositions. Omega-3-fatty acids are anti-inflammatory compounds which act as competitive inhibitors of the arachidonic acid cascade. The omega-3-fatty acids are precursors to the synthesis of prostaglandins which function in mammals to regulate inflammation. See e.g., Burger, U.S. Pat. No. 5,843,919, issued Dec. 1, 1998.

The omega-3-fatty acid optionally utilized herein may be any omega-3-fatty acid or combination of omega-3-fatty acids. Non-limiting examples of omega-3-fatty acids which are suitable for use herein include eicosapentaenoic acid (also known as EPA), docosahexaenoic acid (also known as DHA), and mixtures thereof.

Optionally, the omega-3-fatty acid, as well as all other oil soluble components described herein, can be added to the present compositions via an emulsion and/or encapsulation. Additionally, in essentially dry compositions, the omega-3-fatty acid may be spray dried according to commonly known techniques.

Wherein one or more omega-3-fatty acids is utilized in the present compositions, the ratio of the first component herein and the omega-3-fatty acids is often important for optimization of health benefits, particularly joint health benefits, bone health benefits, and anti-inflammation. Preferably, the ratio of the first component to the total omega-3-fatty acid(s) present in the composition (on a weight to weight basis) is from about 95:5 to about 5:95, more preferably from about 75:25 to about 25:75, most preferably from about 60:40 to about 40:60. The dosage of omega-3-fatty acid(s) included in the composition is therefore preferably administered according to these guidelines. Typical dosage levels of the first component has been detailed herein above.

Bracers

As is commonly known in the art, bracers can be obtained by extraction from a natural source or can be synthetically produced. Non-limiting examples of bracers include methylxanthines, e.g., caffeine, theobromine, and theophylline. Additionally, numerous other xanthine derivatives have been isolated or synthesized, which may be utilized as a bracer in the compositions herein. See e.g., Bruns, Biochemical Pharmacology, Vol. 30, pp. 325-333 (1981) which describes, inter alia, xanthine, 9-methyl xanthine, 7-methyl xanthine, 3-methyl xanthine, 3,7-dimethyl xanthine, 8-chloromethyl-3,7-dimethyl xanthine, 8-hydroxymethyl-3,7-dimethyl xanthine, 3,7-diethyl xanthine, 3,7-bis-(2-hydroxyethyl) xanthine, 3-propyl-7-(dimethylaminoethyl) xanthine, 1-methyl xanthine, 1,9-dimethyl xanthine, 1-methyl-8-methylthio xanthine, 8-phenyl-1-methyl xanthine, 1,7-dimethyl xanthine, 1,7-dimethyl-8-oxo xanthine, 1,3-dimethyl xanthine, 1,3,9-trimethyl xanthine, 8-fluoro theophylline, 8-chloro theophylline, 8-bromo theophylline, 8-thio theophylline, 8-methylthio theophylline, 8-ethylthio theophylline, 8-nitro theophylline, 8-methylamino theophylline, 8-dimethylamino theophylline, 8-methyl theophylline, 8-ethyl theophylline, 8-propyl theophylline, 8-cyclopropyl theophylline, theophylline-8-propionate (ethyl ester), 8-benzyl theophylline, 8-cyclopentyl theophylline, 8-cyclohexyl theophylline, 8-(3-indolyl) theophylline, 8-phenyl theophylline, 9-methyl-8-phenyl theophylline, 8-(p-chlorophenyl) theophylline, 8-(p-bromophenyl) theophylline, 8-(p-methoxyphenyl) theophylline, 8-(p-nitrophenyl) theophylline, 8-(p-dimethylaminophenyl) theophylline, 8-(p-methylphenyl) theophylline, 8-(3,4-dichlorophenyl) theophylline, 8-(m-nitrophenyl) theophylline, 8-(o-nitrophenyl) theophylline, 8-(o-carboxyphenyl) theophylline, 8-(1 -naphthyl) theophylline, 8-(2,6-dimethyl-4-hydroxyphenyl) theophylline, 7-methoxy-8-phenyl theophylline, 1,3,7-trimethyl xanthine, S-chloro caffeine, S-oxo caffeine, S-methoxy caffeine, S-methylamino caffeine, 8-diethylamino caffeine, 8-ethyl caffeine, 7-ethyl theophylline, 7-(2-chloroethyl) theophylline, 7-(2-hydroxyethyl) theophylline, 7-(carboxymethyl) theophylline, 7-(carboxymethyl) theophylline (ethyl ester), 7-(2-hydroxypropyl) theophylline, 7-(2,3-dihydroxypropyl) theophylline, 7-b-D-ribofuranosyl theophylline, 7-(glycero-pent-2-enopyranosyl) theophylline, 7-phenyl theophylline, 7,8-diphenyl theophylline, 1-methyl-3,7-diethyl xanthine, 1-methyl-3-isobutyl xanthine, 1-ethyl-3,7-dimethyl xanthine, 1,3-diethyl xanthine, 1,3,7-triethyl xanthine, 1-ethyl-3-propyl-7-butyl-8-methyl xanthine, 1,3-dipropyl xanthine, 1,3-diallyl xanthine, 1-butyl-3,7-dimethyl xanthine, 1-hexyl-3,7-dimethyl xanthine, and 1-(5-oxohexyl)-3,7-dimethyl xanthine.

Additionally, one or more of these bracers are present in, for example, coffee, tea, kola nut, cacao pod, mate', yaupon, guarana paste, and yoco. Natural plant extracts are the preferred sources of bracers as they may contain other compounds that delay the bioavailability of the bracer thus they may provide mental refreshment and alertness without tension or nervousness.

The most preferred methylxanthine is caffeine. Caffeine may be obtained from the aforementioned plants and their waste or, alternatively, may be synthetically prepared. Preferred botanical sources of caffeine which may be utilized as a complete or partial source of caffeine include green tea, guarana, mate', black tea, cola nuts, cocoa, and coffee. As used herein, green tea, guarana, coffee, and mate' are the most preferred botanical sources of caffeine, most preferably green tea, guarana, and coffee. Mate' may have the additional benefit of an appetite suppressing effect and may be included for this purpose as well. The total amount of caffeine, in any embodiment of the present invention, includes the amount of caffeine naturally present in the tea extract, flavoring agent, botanical and any other components, as well as any added caffeine.

Any bracer utilized herein is preferably present in physiologically relevant amounts, which means that the sources used in the practice of this invention provide a safe and effective quantity to achieve the desired mental alertness.

Wherein a bracer is utilized in the present compositions, such compositions will preferably comprise from about 0.0005% to about 1%, more preferably from about 0.003% to about 0.5%, still more preferably from about 0.003% to about 0.2%, even more preferably from about 0.005% to about 0.05%, and most preferably from about 0.005% to about 0.02% of a bracer, by weight of the composition. Of course, as the skilled artisan will comprehend, the actual amount of bracer added will depend its biological effect, for example, effect of mental alertness on the consumer.

In all of the present compositions, the total amount of bracer includes any added bracer as well as any bracer naturally present in any other component of the present invention.

Flavanols

Flavanols are natural substances present in a variety of plants (e.g., fruits, vegetables, and flowers). The flavanols which may be utilized in the present invention can be extracted from, for example, fruit, vegetables, green tea or other natural sources by any suitable method well known to those skilled in the art. For example, extraction with ethyl acetate or chlorinated organic solvents is a common method to isolate flavanols from green tea. Flavanols may be extracted from either a single plant or mixtures of plants. Many fruits, vegetables, and flowers contain flavanols but to a lesser degree relative to green tea. Plants containing flavanols are known to those skilled in the art. Examples of the most common flavanols which are extracted from tea plants and other members of the Catechu gambir (Uncaria family) include, for example, catechin, epicatechin, gallocatechin, epigallocatechin, epicatechin gallate, and epigallocatechin gallate.

The flavanols utilized in all compositions of the present invention can be in the form of a tea extract. The tea extract can be obtained from the extraction of unfermented teas, fermented teas, partially fermented teas, and mixtures thereof. Preferably, the tea extracts are obtained from the extraction of unfermented and partially fermented teas. The most preferred tea extracts are obtained from green tea. Both hot and cold extracts can be used in the present invention. Suitable methods for obtaining tea extracts are well known. See e.g., Ekanayake, U.S. Pat. No. 5,879,733, issued Mar. 9, 1999; Tsai, U.S. Pat. No. 4,935,256, issued June, 1990; Lunder, U.S. Pat. No. 4,680,193, issued July, 1987; and Creswick, U.S. Pat. No. 4,668,525, issued May 26, 1987.

The preferred source of flavanols in the compositions of the present invention is green tea. Wherein green tea, and in particular the flavanols present in green tea, are incorporated into the beverage, the present inventors have discovered that the flavanols are at least partially responsible for delaying the bioavailability of bracers, which contributes to the reduction and/or elimination of nervousness and tension typically associated with such bracers.

Alternatively, these same flavanols may be prepared by synthetic or other appropriate chemical methods and incorporated into the present compositions. Flavanols, including catechin, epicatechin, and their derivatives are commercially available.

The amount of flavanols in the compositions of the present invention can vary. However, wherein one or more flavanols are utilized, preferably from about 0.001% to about 5%, more preferably from about 0.001% to about 2%, even more preferably from about 0.01% to about 1%, and most preferably from about 0.01% to about 0.05% of one or more flavanols is utilized, by weight of the composition.

In all of the embodiments of the present invention, the total amount of flavanols includes any added flavanols as well as any flavanols naturally present in any other component of the present invention.

Milk Solids and Other Proteins

One or more milk solids may also optionally be included in the compositions of the present invention. As used herein, milk solids means milk from one or more mammals or a plant-derived milk, and includes, for example, fermented milk, lactic acid beverages obtained by lactic acid fermentation or otherwise acidified, sterilized milk base, liquid milk, and milk products such as skim milk powder or whole milk powder or other powdered forms of milk. As used herein, milk solids means the solids content or dry matter of milk base.

Wherein one or more milk solids is utilized, the desired total level of milk solids, calculated on a milk solids basis for the compositions of the present invention, is typically from about 0.001% to about 15%, preferably from about 0.005% to about 10%, and most preferably from about 0.1% to about 5%.

Other proteins, such as soy, whey, casseinates, including isolates of the foregoing, may be utilized in the present compositions. The level of each of these proteins will vary, and may be readily determined by the ordinarily skilled artisan.

Soluble Fibers

One or more soluble fibers may also optionally be included in the compositions of the present invention to provide, for example, nutritive benefits. Soluble fibers which can be used singularly or in combination in all embodiments of the present invention include but are not limited to pectins, psyllium, guar gum, xanthan gum, alginates, gum arabic, fructo-oligosaccbarides, inulin, agar, and carrageenan. Preferred among these soluble fibers are at least one of guar gum, xanthan, and carrageenan, most preferably at least one of guar gum and xanthan. These soluble fibers may also serve as stabilizing agents in the various embodiments of this invention.

Particularly preferred soluble fibers for use herein are glucose polymers, preferably those which have branched chains. Preferred among these soluble fibers is one marketed under the trade name Fibersol2, commercially available from Matsutani Chemical Industry Co., Itami City, Hyogo, Japan.

Pectin and fructo-oligosaccharides are also preferred soluble fibers herein. Even more preferably, pectin and fructo-oligosaccharides are used in combination. The preferred ratio of pectin to fructo-oligosaccharide is from about 3:1 to about 1:3, by weight of the composition. The preferred pectins have a degree of esterification higher than about 65%.

The preferred fructo-oligosaccharides are a mixture of fructo-oligosaccharides composed of a chain of fructose molecules linked to a molecule of sucrose. Most preferably, they have a nystose to kestose to fructosyl-nystose ratio of about 40:50:10, by weight of the composition. Preferred fructo-oligosaccharides may be obtained by enzymatic action of fructosyltransferase on sucrose such as those which are, for example, commercially available from Beghin-Meiji Industries, Neuilly-sur-Seine, France.

Preferred pectins are obtained by hot acidic extraction from citrus peels and may be obtained, for example, from Danisco Co., Braband, Denmark.

Wherein a soluble fiber is utilized, the desired total level of soluble dietary fiber for the compositions of the present invention is from about 0.01% to about 15%, preferably from about 0.1% to about 5%, more preferably from about 0.1% to about 3%, and most preferably from about 0.2% to about 2%, by weight of the composition. The total amount of soluble dietary fiber includes any added soluble dietary fiber as well as any soluble dietary fiber naturally present in any other component of the present invention.

Sweeteners

The compositions of the present invention can, and typically will, contain an effective amount of one or more sweeteners, including carbohydrate sweeteners and natural and/or artificial no/low calorie sweeteners. The amount of the sweetener used in the beverages of the present invention typically depends upon the particular sweetener used and the sweetness intensity desired. For no/low calorie sweeteners, this amount varies depending upon the sweetness intensity of the particular sweetener.

The compositions of the present invention can be sweetened with any of the carbohydrate sweeteners, preferably monosaccharides and/or disaccharides. Sweetened beverages will typically comprise from about 0.1% to about 20%, most preferably from about 6 to about 14%, sweetener. These sugars can be incorporated into the beverages in solid or liquid form but are typically, and preferably, incorporated as a syrup, most preferably as a concentrated syrup such as high fructose corn syrup. For purposes of preparing beverages of the present invention, these sugar sweeteners can be provided to some extent by other components of the beverage such as, for example, the fruit juice component and/or flavors.

Preferred sugar sweeteners for use in beverage products of the present invention are sucrose, fructose, glucose, and mixtures thereof, particularly sucrose and fructose. Fructose can be obtained or provided as liquid fructose, high fructose corn syrup, dry fructose or fructose syrup, but is preferably provided as high fructose corn syrup. High fructose corn syrup (HFCS) is commercially available as HFCS-42, HFCS-55 and HFCS-90, which comprise 42%, 55% and 90%, respectively, by weight of the sugar solids therein, as fructose.

Non-limiting examples of no/low-caloric sweeteners include sorbitol, mannitol, xylitol, erythritol, malitol, maltose, lactose, fructooligosaccharides, lo han guo, stevioside, acesulfame, aspartame, sucralose, saccharin, xylose, arabinose, levulose, isomalt, ribose, and mixtures thereof. Preferred among these examples include xylitol, erythritol, fructooligosaccharides, lo han guo, stevioside, acesulfame, sucralose, and mixtures thereof. Even more preferred among these examples include erythritol, fructooligosaccharides, lo han guo, acesulfame, sucralose, and mixtures thereof.

Naturally occurring sweeteners or their purified extracts, such as stevioside, the protein sweetener thaumatin, lo han guo (disclosed in, for example, Fischer et al., U.S. Pat. No. 5,433,965, issued Jul. 18, 1995), and the like can be utilized as the sweetener herein.

The preferred fructooligosaccharides are a mixture of fructooligosaccharides composed of a chain of fructose molecules linked to a molecule of sucrose. Most preferably, these fructooligosaccharides have a nystose to kestose to fructosyl-nystose ratio of about 40:50:10, by weight of the composition. Preferred fructooligosaccharides may be obtained by enzymatic action of fructosyltransferase on sucrose such as those which are, for example, commercially available from Beghin-Meiji Industries, Neuilly-sur-Seine, France.

Other non-limiting examples of such sweeteners include polyols, which are preferred due to their ability to provide the bulk of sugar, but without the calories and glycemic contribution of sugar. Thus, polyols are particularly useful for controlling increases in blood glucose and insulin levels. Non-limiting examples of well-known polyols for such use as sweetening agents include erythritol, mannitol, isomalt, lactitol, maltitol, sorbitol, and xylitol.

Erythritol is a particularly preferred sweetening agent for use herein. Erythritol is a polyol which is commonly used as a bulk sweetener in reduced calorie foods. Erythritol provides about 70% of the “sweetness” relative to sucrose, and about 5% of the calories relative to sucrose. In the United States, erythritol is typically labeled to provide approximately 0.2 calories per gram. Similarly, mannitol, isomalt, lactitol, maltitol, sorbitol, and/or xylitol may be utilized in the present compositions to provide the bulk of traditional sugars with exceedingly less caloric intake and blood sugar contribution.

Sucralose is also particularly preferred for use herein. Sucralose has little to no effect on sugar or carbohydrate metabolism, blood glucose elevation, or insulin production. Sucralose is commercially available from, for example, McNeil Specialty Products Company, New Brunswick, N.J.

Non-limiting examples of other non-caloric sweetening agents include aspartame, saccharin, cyclamates, acesulfame K, L-aspartyl-L-phenylalanine lower alkyl ester sweeteners, L-aspartyl-D-alanine amides such as, for example, those disclosed in Brennan et al., U.S. Pat. No. 4,411,925, issued 1983, L-aspartyl-D-serine amides such as, for example, those disclosed in Brennan et al., U.S. Pat. No. 4,399,163, issued 1983, L-aspartyl-hydroxymethyl alkane amide sweeteners such as, for example, those disclosed in Brand, U.S. Pat. No. 4,338,346, issued 1982, L-aspartyl-l-hydroxyethylalkane amide sweeteners such as, for example, those disclosed in Rizzi, U.S. Pat. No. 4,423,029, issued 1983, glycyrrhizins, and synthetic alkoxy aromatics.

The amount of sweetener used in the compositions of the present invention typically depends upon the particular sweetener used and the sweetness intensity desired. Typically, the present compositions comprise from about 0.00001% to about 75% total sweetener, by weight of the composition. Dry beverage compositions (i.e., those which are suitable for dilution to provide a concentrate or ready-to-drink beverage composition) will typically comprise from about 0.0001% to about 75%, more preferably from about 5% to about 65%, even more preferably from about 10% to about 60%, and most preferably from about 20% to about 55% total sweetener, all by weight of the composition (i.e., the dry beverage composition). Concentrates which are suitable for dilution to provide a ready-to-drink beverage composition will typically comprise from about 0.0001% to about 75%, more preferably from about 1% to about 50%, even more preferably from about 2% to about 40%, and most preferably from about 5% to about 30% total sweetener, all by weight of the composition (i.e., the concentrate). Ready-to-drink beverage compositions will typically comprise from about 0.0001% to about 50%, more preferably from about 0.001% to about 25%, even more preferably from about 0.01% to about 10%, and most preferably from about 0.25% to about 5% total sweetener, all by weight of the composition (i.e., the ready-to-drink beverage composition). Wherein mixtures of sweeteners are utilized, the relative weight percentages of each sweetener collectively provides the amount of total sweetener present in the composition.

Nutrients

As discussed in considerable detail above, the present compositions comprise a second component which will comprise a cation component selected from calcium, potassium, and magnesium, which are each nutritive minerals. The compositions herein may optionally, but preferably, be fortified further with one or more nutrients, especially one or more vitamins and/or minerals. The U.S. Recommended Daily Intake (USRDI) for vitamins and minerals are defined and set forth in the Recommended Daily Dietary Allowance-Food and Nutrition Board, National Academy of Sciences-National Research Council.

Unless otherwise specified herein, wherein a given mineral is present in the composition, the composition typically comprises at least about 1%, preferably at least about 5%, more preferably from about 10% to about 200%, even more preferably from about 40% to about 150%, and most preferably from about 60% to about 125% of the USRDI of such mineral. Unless otherwise specified herein, wherein a given mineral is present in the composition, the composition comprises at least about 1%, preferably at least about 5%, more preferably from about 10% to about 200%, even more preferably from about 20% to about 150%, and most preferably from about 25% to about 120% of the USRDI of such vitamin.

Non-limiting examples of such further vitamins and minerals, include niacin, thiamin, folic acid, pantothenic acid, biotin, vitamin A, vitamin C, vitamin B_{2,} vitamin B₃, vitamin B₆, vitamin B₁₂, vitamin D, vitamin E, vitamin K, iron, zinc, copper, phosphorous, iodine, chromium, molybdenum, and fluoride. Preferably, wherein a further vitamin or mineral is utilized the vitamin or mineral is selected from niacin, thiamin, folic acid, iodine, vitamin A, vitamin C, vitamin B_{6,} vitamin B₁₂, vitamin D, vitamin E, iron, zinc, and calcium. Preferably, at least one vitamin is selected from vitamin C, vitamin B_{6,} vitamin B₁₂, vitamin E, pantothenic acid, niacin, and biotin. Also preferably, the composition comprises vitamin C and one or more other vitamins selected from vitamin B₆, vitamin B_{12,} vitamin E, pantothenic acid, niacin, and biotin.

Commercially available vitamin A sources may also be included in the present compositions. As used herein, “vitamin A” includes, but is not limited to, vitamin A (retinol), β-carotene, retinol palmitate, and retinol acetate. The vitamin A may be in any form, for example, an oil, beadlets, or encapsulated. Wherein vitamin A is present in the compositions herein, the product comprises at least about 1%, preferably at least about 5%, more preferably from about 10% to about 200%, even more preferably from about 15% to about 150%, and most preferably from about 20% to about 120% of the USRDI of such vitamin. Wherein vitamin A is present in the compositions herein, it is especially preferred to include about 25% of the USRDI of vitamin A. The quantity of vitamin A to be added is dependent on processing conditions and the amount of vitamin A deliver desired after storage. Preferably, wherein vitamin A is included within the present compositions, the compositions comprise from about 0.0001% to about 0.2%, more preferably from about 0.0002% to about 0.12%, also preferably from about 0.0003% to about 0.1%, even more preferably from about 0.0005% to about 0.08%, and most preferably from about 0.001% to about 0.06% of vitamin A, by weight of the product.

Commercially available sources of vitamin B₂ (also known as riboflavin) may be utilized in the present compositions. Wherein vitamin B₂ is present in the compositions herein, the product comprises at least about 1%, preferably at least about 5%, more preferably from about 5% to about 200%, even more preferably from about 10% to about 150%, and most preferably from about 10% to about 120% of the USRDI of such vitamin. Wherein vitamin B₂ is present in the compositions herein, it is especially preferred to include from about 15% to about 35% of the USRDI of vitamin B₂.

Vitamin C (ascorbic acid) is a particularly preferred optional ingredient for use herein. Without intending to be limited by theory, it is believed that vitamin C may be utilized to enhance the benefits herein, by serving as a co-factor for the enzyme which cross-links collagen.

Encapsulated ascorbic acid and edible salts of ascorbic acid can also be used. Wherein vitamin C is present in the compositions herein, the product comprises at least about 1%, preferably at least about 5%, more preferably from about 10% to about 200%, even more preferably from about 20% to about 150%, and most preferably from about 25% to about 120% of the USRDI of such vitamin. Wherein vitamin C is present in the compositions herein, it is especially preferred to include about 100% of the USRDI of vitamin C. The quantity of vitamin C to be added is dependent on processing conditions and the amount of vitamin C deliver desired after storage. Preferably, wherein vitamin C is included within the present compositions, the compositions comprise from about 0.005% to about 0.2%, more preferably from about 0.01% to about 0.12%, also preferably from about 0.02% to about 0.1%, even more preferably from about 0.02% to about 0.08%, and most preferably from about 0.03% to about 0.06% of vitamin C, by weight of the product.

Nutritionally supplemental amounts of other vitamins which may be incorporated herein include, but are not limited to, vitamins B₆ and B_{12,} folic acid, niacin, pantothenic acid, folic acid, vitamin D, and vitamin E. Wherein the product comprises one of these vitamins, the product preferably comprises at least 5%, preferably at least 25%, and most preferably at least 35% of the USRDI for such vitamin.

Minerals which may optionally be included (as additional to the second component) in the compositions herein are, for example, calcium, manganese, magnesium, boron, zinc, iodine, iron, and copper. Minerals may be, for example, salts, chelated, complexed, or in colloidal form.

Manganese is a particularly preferred mineral for use herein, as this mineral is involved in the synthesis of glycosaminoglycans, collagen, and glycoproteins. Additionally manganese deficiencies can lead to abnormal bone growth, inflamed joints, bone loss, and arthritis. Manganese ascorbate is a particularly preferred form of manganese for use herein. Typical manganese dosages range from about 0 mg to about 1000 mg, more preferably from about 50 mg to about 950 mg, and most preferably from about 50 mg to about 250 mg for a human or large mammal (e.g., horse).

Boron is a particularly preferred mineral for use herein, as this mineral is necessary for osteocalcin formation in bone.

Any soluble salt of these minerals suitable for inclusion edible compositions can be used, for example, zinc chloride, zinc sulfate, copper sulfate, copper gluconate, and copper citrate.

Commercial sources of iodine, preferably as an encapsulated iodine may be utilized herein. Other sources of iodine include iodine-containing salts, e.g., sodium iodide, potassium iodide, potassium iodate, sodium iodate, or mixtures thereof. These salts may be encapsulated.

Iron may also be utilized in the compositions and methods of the present invention. Acceptable forms of iron are well-known in the art. The amount of iron compound incorporated into the product will vary widely depending upon the level of supplementation desired in the final product and the targeted consumer. Iron fortified compositions of the present invention typically contain from about 5% to about 100%, preferably from about 15% to about 50%, and most preferably about 20% to about 40% of the USRDI for iron.

Ferrous iron is typically better utilized by the body than ferric iron. Highly bioavailable ferrous salts that can be used in the ingestible compositions of the present invention are ferrous sulfate, ferrous fumarate, ferrous succinate, ferrous gluconate, ferrous lactate, ferrous tartarate, ferrous citrate, ferrous amino acid chelates, as well as mixtures of these ferrous salts. While ferrous iron is typically more bioavailable, certain ferric salts can also provide highly bioavailable sources of iron. Highly bioavailable ferric salts that can be used in the food or beverage compositions of the present invention are ferric saccharate, ferric ammonium citrate, ferric citrate, ferric sulfate, as well as mixtures of these ferric salts. Combinations or mixtures of highly bioavailable ferrous and ferric salts can be used in these edible mixes and ready-to-serve beverages. The preferred sources of highly bioavailable iron are ferrous fumarate and ferrous amino acid chelates.

Ferrous amino acid chelates particularly suitable as highly bioavailable iron sources for use in the present invention are those having a ligand to metal ratio of at least 2:1. For example, suitable ferrous amino acid chelates having a ligand to metal mole ratio of two are those of formula:

Fe(L)₂

where L is an alpha amino acid, dipeptide, tripeptide, or quadrapeptide ligand. Thus, L can be any ligand which is a naturally occurring alpha amino acid selected from alanine, arginine, asparagine, aspartic acid, cysteine, cystine, glutamine, glutamic acid, glycine, histidine, hydroxyproline, isoleucine, leucine, lysine, methionine, omithine, phenylalanine, proline, serine, threonine, tryptophan, tyrosine, and valine; or dipeptides, tripeptides, or quadrapeptides formed by any combination of these alpha amino acids. See e.g., Ashmead et al., U.S. Pat. No. 4,863,898, issued Sept. 5, 1989; Ashmead, U.S. Pat. No. 4,830,716, issued May 16, 1989; and Ashmead, U.S. Pat. No. 4,599,152, issued Jul. 8, 1986, all of which are incorporated by reference. Particularly preferred ferrous amino acid chelates are those where the reacting ligands are glycine, lysine, and leucine. Most preferred is the ferrous amino acid chelate sold under the mark Ferrochel® (Albion Laboratories, Salt Lake City, Utah) wherein the ligand is glycine.

In addition to these highly bioavailable ferrous and ferric salts, other sources of bioavailable iron can be included in the food and beverage compositions of the present invention. Other sources of iron particularly suitable for fortifying compositions of the present invention included certain iron-sugar-carboxylate complexes. In these iron-sugar-carboxylate complexes, the carboxylate provides the counterion for the ferrous (preferred) or ferric iron. The overall synthesis of these iron-sugar-carboxylate complexes involves the formation of a calcium-sugar moiety in aqueous media (for example, by reacting calcium hydroxide with a sugar, reacting the iron source (such as ferrous ammonium sulfate) with the calcium-sugar moiety in aqueous media to provide an iron-sugar moiety, and neutralizing the reaction system with a carboxylic acid (the “carboxylate counterion”) to provide the desired iron-sugar- carboxylate complex. Sugars that can be used to prepare the calcium-sugar moiety include any of the ingestible saccharidic materials, and mixtures thereof, such as glucose, sucrose and fructose, mannose, galactose, lactose, maltose, and the like, with sucrose and fructose being the more preferred. The carboxylic acid providing the “carboxylate counterion” can be any ingestible carboxylic acid such as citric acid, malic acid tartaric acid, lactic acid, succinic acid, propionic acid, etc., as well as mixtures of these acids.

These iron-sugar-carboxylate complexes can be prepared in the manner described in, e.g., Nakel et al., U.S. Pat. Nos. 4,786,510 and 4,786,518, issued Nov. 22, 1988, both of which are incorporated by reference. These materials are referred to as “complexes”, but they may exist in solution as complicated, highly hydrated, protected colloids; the term “complex” is used for the purpose of simplicity.

Zinc may also be utilized in the compositions and methods of the present invention. Acceptable forms of zinc are well-known in the art. Zinc fortified compositions of the present invention typically contain from about 5% to about 100%, preferably from about 15% to about 50%, and most preferably about 25% to about 45% of the USRDI for zinc. The zinc compounds which can be used in the present invention can be in any of the commonly used forms such as, e.g., zinc sulfate, zinc chloride, zinc acetate, zinc gluconate, zinc ascorbate, zinc citrate, zinc aspartate, zinc picolinate, amino acid chelated zinc, and zinc oxide. Zinc gluconate and amino acid chelated zinc are particularly preferred.

Flavoring Agents

One or more flavoring agents are recommended for the embodiments of the present invention in order to enhance their palatability. Any natural or synthetic flavor agent can be used in the present invention. For example, one or more botanical and/or fruit flavors may be utilized herein. As used herein, such flavors may be synthetic or natural flavors.

Particularly preferred fruit flavors are exotic and lactonic flavors such as, for example, passion fruit flavors, mango flavors, pineapple flavors, cupuacu flavors, guava flavors, cocoa flavors, papaya flavors, peach flavors, and apricot flavors. Besides these flavors, a variety of other fruit flavors can be utilized such as, for example, apple flavors, citrus flavors, grape flavors, raspberry flavors, cranberry flavors, cherry flavors, grapefruit flavors, and the like. These fruit flavors can be derived from natural sources such as fruit juices and flavor oils, or may alternatively be synthetically prepared.

Preferred botanical flavors include, for example, tea (preferably black and green tea, most preferably green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardimom, nutmeg, cassia bark, buchu, cinnamon, jasmine, haw, chrysanthemum, water chestnut, sugar cane, lychee, bamboo shoots, vanilla, coffee, and the like. Preferred among these is tea, guarana, ginseng, ginko, and coffee. In particular, the combination of tea flavors, preferably green tea or black tea flavors (preferably green tea), optionally together with fruit flavors has an appealing taste. In another preferred embodiment, coffee is included within the present compositions. A combination of green tea and coffee in the present compositions is often preferred.

The flavor agent can also comprise a blend of various flavors. If desired, the flavor in the flavoring agent may be formed into emulsion droplets which are then dispersed in the beverage composition or concentrate. Because these droplets usually have a specific gravity less than that of water and would therefore form a separate phase, weighting agents (which can also act as clouding agents) can be used to keep the emulsion droplets dispersed in the beverage composition or concentrate. Examples of such weighting agents are brominated vegetable oils (BVO) and resin esters, in particular the ester gums. See L.F. Green, Developments in Soft Drinks Technology, Vol. 1, Applied Science Publishers Ltd., pp. 87-93 (1978) for a further description of the use of weighting and clouding agents in liquid beverages. Typically the flavoring agents are conventionally available as concentrates or extracts or in the form of synthetically produced flavoring esters, alcohols, aldehydes, terpenes, sesquiterpenes, and the like.

Coloring Agent

Small amounts of one or more coloring agents may be utilized in the compositions of the present invention. FD&C dyes (e.g., yellow #5, blue #2, red # 40) and/or FD&C lakes are preferably used. By adding the lakes to the other powdered ingredients, all the particles, in particular the colored iron compound, are completely and uniformly colored and a uniformly colored beverage mix is attained. Preferred lake dyes which may be used in the present invention are the FDA-approved Lake, such as Lake red #40, yellow #6, blue #1, and the like. Additionally, a mixture of FD&C dyes or a FD&C lake dye in combination with other conventional food and food colorants may be used. Riboflavin and b-carotene may also be used. Additionally, other natural coloring agents may be utilized including, for example, fruit, vegetable, and/or plant extracts such as grape, black currant, aronia, carrot, beetroot, red cabbage, and hibiscus.

The amount of coloring agent used will vary, depending on the agents used and the intensity desired in the finished product. The amount can be readily determined by one skilled in the art. Generally, if utilized, the coloring agent should be present at a level of from about 0.0001% to about 0.5%, preferably from about 0.001% to about 0.1%, and most preferably from about 0.004% to about 0.1%, by weight of the composition.

Preservatives

Optionally, one or more preservatives may additionally be utilized herein. Preferred preservatives include, for example, sorbate, benzoate, and polyphosphate preservatives.

Preferably, wherein a preservative is utilized herein, one or more sorbate or benzoate preservatives (or mixtures thereof) are utilized. Sorbate and benzoate preservatives suitable for use in the present invention include sorbic acid, benzoic acid, and salts thereof, including (but not limited to) calcium sorbate, sodium sorbate, potassium sorbate, calcium benzoate, sodium benzoate, potassium benzoate, and mixtures thereof. Sorbate preservatives are particularly preferred. Potassium sorbate is particularly preferred for use in the present invention.

Wherein a composition comprises a preservative, the preservative is preferably included at levels from about 0.0005% to about 0.5%, more preferably from about 0.001% to about 0.4% of the preservative, still more preferably from about 0.001% to about 0.1%, even more preferably from about 0.001% to about 0.05%, and most preferably from about 0.003% to about 0.03% of the preservative, by weight of the composition. Wherein the composition comprises a mixture of one or more preservatives, the total concentration of such preservatives is preferably maintained within these ranges.

Acidulants

If desired, the present compositions may optionally comprise one or more acidulants. An amount of an acidulant may be used to maintain the pH of the composition. Compositions of the present invention preferably have a pH of from about 2 to about 7, more preferably from about 2 to about 5, even more preferably from about 3 to about 5, and most preferably from about 3.5 to about 4.5. Beverage acidity can be adjusted to and maintained within the requisite range by known and conventional methods, e.g., the use of one or more of the aforementioned acidulants. Typically, acidity within the above recited ranges is a balance between maximum acidity for microbial inhibition and optimum acidity for the desired beverage flavor.

Organic as well as inorganic edible acids may be used to adjust the pH of the beverage, and may be added additional to the acid serving as part of the second component herein. The acids can be present in their undissociated form or, alternatively, as their respective salts, for example, potassium or sodium hydrogen phosphate, potassium or sodium dihydrogen phosphate salts. The preferred acids are edible organic acids which include citric acid, malic acid, fumaric acid, adipic acid, phosphoric acid, gluconic acid, tartaric acid, ascorbic acid, acetic acid, phosphoric acid or mixtures thereof. The most preferred acids are citric and malic acids.

The acidulant can also serve as an antioxidant to stabilize beverage components. Examples of commonly used antioxidant include but are not limited to ascorbic acid, EDTA (ethylenediaminetetraacetic acid), and salts thereof.

Emulsifiers and Oils

One or more emulsifiers and/or oils may also be included in the present compositions for texture and opacity purposes. Typical emulsifiers and oils useful herein include, for example, mono-di glycerides, lecithin, pulp, cotton seed oil, and vegetable oil.

Thickeners

One or more thickeners may be optionally added to the present compositions to, for example, provide control of viscosity and / or texture. Various thickeners are well-known in the art. Non-limiting examples of thickeners include cellulose compounds, gum ghatti, modified gum ghatti, xanthan gum, tragacanth gum, guar gum, gellan gum, locust bean gum, pectin, and mixtures thereof. See e.g., Kupper et al., U.S. Pat. No. 4,705,691, issued Nov. 10, 1987. Particularly preferred for use herein include xanthan gum, gellan gum, guar gum, and cellulose compounds.

Cellulose compounds are widely known in the art. Cellulose compounds are typically anionic polymers derived from cellulose. Non-limiting examples of cellulose compounds utilized herein include carboxymethylcellulose, methylcellulose, and hydroxyethylcellulose, hydroxypropylcellulose. The most preferred cellulose compound for use in the present compositions is carboxymethylcellulose, particularly sodium carboxymethylcellulose. Non-limiting examples of cellulose compounds include sodium carboxymethylcellulose (commercially available as Aqualon® 7HOF from Hercules, Inc., Wilmington, Del.

When present, the thickener is typically utilized in the present compositions at levels preferably from about 0.00001% to about 10%, more preferably from about 0.00001% to about 5%, still more preferably from about 0.00001% to about 1%, even more preferably from about 0.01% to about 0.2%, and most preferably from about 0.02% to about 0.05%, by weight of the composition.

Water

Water may be, and preferably is, included in the present compositions. Water is most preferably included in the beverage composition embodiments of the present invention. Wherein water is included within the compositions herein, the compositions preferably comprise at least about 20% water, more preferably at least about 40% water, still more preferably at least about 50% water, even more preferably at least about 75% water, and most preferably at least about 80% water. Still further, ready-to-drink beverage compositions will typically comprise from at least about 80% water to about 99.9% water.

Carbonation Component

Carbon dioxide can be introduced into the water which is mixed with a beverage concentrate or into the beverage composition after dilution to achieve carbonation. The carbonated beverage can be placed into a container, such as a bottle or can, and then sealed. Any conventional carbonation methodology may be utilized to make carbonated beverage compositions of this invention. The amount of carbon dioxide introduced into the beverage will depend upon the particular flavor system utilized and the amount of carbonation desired.

### Methods of Making

The present compositions are made according to methods which will be well known by the ordinarily skilled artisan. To illustrate, the compositions of the present invention may be prepared by dissolving, dispersing, or otherwise mixing all components singularly or in suitable combinations together and in water where appropriate, agitating with a mechanical stirrer until all of the ingredients have been solubilized or adequately dispersed. Where appropriate, all separate solutions and dispersed may then be combined. When using tea extracts which typically are pH sensitive, it is important to adjust the desired pH with an acidulant and/or buffer system before adding the tea extracts to the mixture. Wherein a shelf stable composition is desired, the final mixture can optionally, but preferably, be pasteurized or filled aseptically at appropriate process conditions.

In making a beverage composition, a beverage concentrate may optionally be formed first. One method to prepare the concentrate form of the beverage composition would be to start with less than the required volume of water that is used in the preparation of the beverage composition. Another method would be to partially dehydrate the finally prepared beverage compositions to remove only a portion of the water and any other volatile liquids present. Dehydration may be accomplished in accordance with well known procedures, such as evaporation under vacuum. The concentrate can be in the form of a relatively thick liquid. A syrup is typically formed by adding suitable ingredients such as electrolytes or emulsions to the beverage concentrate. The syrup is then mixed with water to form a finished beverage or finished beverage concentrate. The weight ratio of water to syrup is typically from about 1:1 to about 5:1.

Carbon dioxide can be introduced either into the water to be mixed with the beverage concentrate, or into the drinkable beverage composition, to achieve carbonation. The carbonated beverage composition can then be stored in a suitable container and then sealed. Techniques for making and carbonating beverage embodiments of the present invention are described in the following references: L. F. Green (ed.), Developments in Soft Drinks Technology, Vol. 1 (Elsevier, 1978); G. S. Cattell and P. M. Davies, “Preparation and Processing of Fruit Juices, Cordials and Drinks”, Journal of the Society of Dairy Technology; Vol. 38 (1), pp. 21-27, A. H. Varnam and J. P. Sutherland, Beverages—Technology, Chemistry and Microbiology, Chapman Hall, 1994; and A. J. Mitchell (ed.), Formulation and Production of Carbonated Soft Drinks, Blackie and Sons Ltd., 1990.

Dry compositions, or essentially dry composition of the present invention can be prepared by blending the proper amounts and ratios of all the required dry ingredients together. Alternatively, the finally prepared beverage compositions can be dehydrated to give the essentially dry composition of the beverage composition. The essentially dry composition, either as, for example, a powder, granules or tablets, can later be dissolved in a proper amount of water, carbonated or non-carbonated, to make the final drinkable beverage or taken in conjunction with water. Alternatively, dry forms of the present invention may be incorporated in other compositions, including but not limited to cereal bars, breakfast bars, energy bars, and nutritional bars.

Other essentially dry compositions include, for example, tablets, capsules, granules, and dry powders. Tablets may contain suitable binders, lubricants, diluents, disintegrating agents, coloring agents, flavoring agents, flow-inducing agents, and melting agents. Suitable carriers and excipients that may be used to formulate dry fonns of the present invention are described in, for example, Rober, U.S. Pat. No. 3,903,297, issued Sept. 2, 1975. Techniques and compositions for making dry forms useful in the methods of this invention are described in the following references: H. W. Houghton (ed.), Developments in Soft Drinks Technology, Vol. 3, Chapter 6, (Elsevier, 1984); Modern Pharmaceutics, Chapters 9 and 10 (Banker & Rodes (ed.), 1979); Libernan et al., Pharmaceutical Dosage Forms: Tablets (1981); and Ansel, Introduction to Pharmaceutical Dosage Forms, 2nd Ed., (1976).

### Kits of the Present Invention

The compositions of the present invention, including food and beverage compositions, may be utilized in kits as described herein. The kits of the present invention comprise one or more compositions of the present invention together with information which informs a user of the kit, by words, pictures, and/or the like, that use of the kit will provide one or more general health and/or general physiological benefits including, but not limited to, joint health benefits (including relief from, prevention of, and/or inhibition of, arthritis and/or osteoarthritis, as well as enhanced flexibility), bone health benefits (including maintaining and/or building bones), cardiac health, anti-inflammation (e.g., pain relief), refreshment, and nutrition (including specific nutritional benefits).

In a particularly preferred embodiment, the information is printed on a container holding the composition, e.g., a bottle. These preferred kits may be in the form of one bottle containing the composition, or may be obtained as a plurality of bottles each containing the composition. For example, the kits may be obtained as one bottle, or cases of four, six, seven (e.g., a weekly supply), or eight bottles co-packaged together. Additionally, monthly kits may be obtained as cases of, for example, twenty-eight or thirty bottles co-packaged together.

### Methods of the Present Invention

The methods of the present invention comprise orally administering (i.e., through ingestion) a composition of the present invention to a mammal, preferably a human, to provide various health benefits, including joint, bone, cardiac, and anti-inflammation benefits, as well as nutritive and organoleptic benefits. The compositions of the present invention are most preferably ingested by consumers desiring a palatable composition, a means to satisfy between-meal hunger, or as a substitute for ingesting a pill-form such to increase compliance. The compositions are also preferably ingested by consumers who experience joint and/or bone dysfunction or those who desire to maintain current joint and/or bone function (i.e., prophylactic use). The compositions of this invention may also be ingested as a supplement to normal dietetic requirements. Frequency of administration is not limited, however, such administration is typically at least once weekly, more preferably at least 3 times weekly, and most preferably at least once daily.

As used herein, the term “orally administering” with respect to the mammal (preferably, human) means that the mammal ingests or is directed to ingest (preferably, for the purpose of providing joint and/or bone health) one or more compositions of the present invention. Preferably, the composition is a beverage composition. Wherein the mammal is directed to ingest one or more of the compositions, such direction may be that which instructs and/or informs the user that use of the composition may and/or will provide one or more general health and/or general physiological benefits including, but not limited to, joint health, bone health, cardiac health, anti-inflammation, refreshment, satiation, and nutrition. For example, such direction may be oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g, a label present on a package containing the composition). As used herein, “written” means through words, pictures, symbols, and/or other visible descriptors. Such direction need not utilize the actual words used herein, for example, “joint”, “bone”, “human”, or “mammal”, but rather use of words, pictures, symbols, and the like conveying the same or similar meaning are contemplated within the scope of this invention.

### EXAMPLES

The following are non-limiting examples of the present compositions which are prepared utilizing conventional methods. The following examples are provided to illustrate the invention and are not intended to limit the scope thereof in any manner.

### Example 1

An 8 oz. beverage composition is prepared by combining the following components in a conventional manner:

| Component | Wt % |
|---|---|
| Glucosamine Hydrochloride | 0.75 |
| Fructose | 9.3 |
| Thickeners | 0.04 |
| Calcium Hydroxide | 0.24 |
| Malic Acid | 0.22 |
| Citric Acid | 0.56 |
| Fruit Juice Concentrate | 1.65 |
| Natural Flavors | 0.02 |
| Ascorbic Acid | 0.07 |
| Water | quantum satis |

In a particularly preferred example of this beverage composition, approximately 1800 mg of the glucosamine hydrochloride is used in the composition. If needed, the pH of the beverage composition is adjusted to from about 3.7 to about 4.0. Various flavors of the beverage composition may be formulated according to standard techniques, for example, grapefruit and/or cranberry flavors.

### Example 2

A kit comprising the beverage composition of Example I and information describing the benefits of consuming the beverage composition is prepared. The beverage composition is contained within a glass bottle containing language such as “Improves Flexibility”, “Excellent Source of Calcium”, and/or the like. The kit is obtained by a 50-year-old female human and is orally ingested by the female human.

### Example 3

A 4 oz. beverage composition is prepared by combining the following components in a conventional manner:

| Component | Wt % |
|---|---|
| Glucosamine Hydrochloride | 1.6 |
| Fructose | 9.3 |
| Thickeners | 0.04 |
| Calcium Hydroxide | 0.48 |
| Malic Acid | 0.44 |
| Citric Acid | 0.22 |
| Fruit Juice Concentrate | 1.65 |
| Natural Flavors | 0.02 |
| Ascorbic Acid | 0.08 |
| Citric Acid (Beyond that which is used for | 0.35 |
| Second Component) | |
| Water | quantum satis |

If needed, the pH of the beverage composition is adjusted to from about 3.7 to about 4.0. Various flavors of the beverage composition may be formulated according to standard techniques, for example, grapefruit and/or cranberry flavors. If desired, this beverage composition may be further diluted by the consumer prior to ingestion with additional water, or a beverage of the consumer's choice.

### Example 4

A kit comprising the beverage composition of Example 3 and information describing the benefits of consuming the beverage composition is prepared. The beverage composition is contained within a glass bottle containing language such as “Improves Flexibility and Joint Health”, “Excellent Source of Calcium”, and/or the like. The kit is obtained by a 45-year-old female human and is orally ingested by the female human.

### Example 5

A 2 oz. beverage composition is prepared by combining the following components in a conventional manner:

| Component | Wt % |
|---|---|
| Glucosamine Hydrochloride | 3.2 |
| Fructose | 9.3 |
| Thickeners | 0.04 |
| Calcium Hydroxide | 0.98 |
| Malic Acid | 0.88 |
| Citric Acid | 0.44 |
| Fruit Juice Concentrate | 1.65 |
| Natural Flavors | 0.02 |
| Ascorbic Acid | 0.16 |
| Citric Acid (Beyond that which is used for | 0.35 |
| Second Component) | |
| Water | quantum satis |

If needed, the pH of the beverage composition is adjusted to from about 3.7 to about 4.0. Various flavors of the beverage composition may be formulated according to standard techniques, for example, grapefruit and/or cranberry flavors. Preferably, this beverage composition may be further diluted by the consumer prior to ingestion with additional water, or a beverage of the consumer's choice.

### Example 6

A kit comprising the beverage composition of Example 5 and information describing the benefits of consuming the beverage composition is prepared. The beverage composition is contained within a glass bottle containing language such as “Improves Flexibility”, “Excellent Source of Calcium”, and/or the like. The kit is obtained by a 29-year-old athletic female human and is orally ingested by the female human.

### Example 7

A beverage composition, which is a concentrate, is prepared by combining the following components in a conventional manner:

| Component | Wt % |
|---|---|
| Glucosamine Hydrochloride | 3.2 |
| Chondroitin Sulfate | 4.0 |
| Fructose | 18.6 |
| Thickeners | 0.08 |
| Calcium Hydroxide | 0.98 |
| Malic Acid | 0.88 |
| Citric Acid | 0.44 |
| Fruit Juice Concentrate | 3.3 |
| Natural Flavors | 0.04 |
| Ascorbic Acid | 0.16 |
| Citric Acid (Beyond that which is used for | 0.7 |
| Second Component) | |
| Water | quantum satis |

Various flavors of the beverage composition may be formulated according to standard techniques, for example, grapefruit and/or cranberry flavors. A consumer purchases this concentrate and further dilutes the concentrate with approximately 150 g of water, or other desired beverage.

### Example 8

A kit comprising the beverage composition of Example 7 and information describing the benefits of consuming the beverage composition is prepared. The beverage composition is contained within a glass bottle containing language such as “Fights the Pain Associated with Arthritis”, “Excellent Source of Calcium”, and/or the like. The kit is obtained by a 45-year-old female human, further diluted with approximately 150 g of water, and is orally ingested by the female human.

### Example 9

A peanut butter fudge bar (suitable for use as a dessert or snack) is prepared by combining the following components:

| Component | Wt % |
|---|---|
| Glucosamine hydrochloride | 2.1 |
| Sweetened Condensed Milk | 30.6 |
| Calcium carbonate | 1 |
| Citric acid | 0.7 |
| Malic acid | 0.7 |
| Chocolate chips | 26 |
| Creamy peanut butter (commercially available as | 38.9 |
| JIF, from The Procter & Gamble Co., Cincinnati, | |
| OH) | |

In particular, approximately 655 grams of the bar is prepared by mixing the sweetened condensed milk, glucosamine hydrochloride, calcium carbonate, citric acid, and malic acid in a mixing bowl. The chocolate chips and peanut butter are melted together. All of the ingredients are mixed together and placed into a pie dish lined with wax paper. Cool in a refrigerator and cut into approximately 8-9 bars.

### Example 10

A tangy cheese spread (suitable for spreading on a cracker) is prepared by combining the following components:

| Component | Wt % |
|---|---|
| Glucosamine hydrochloride | 6.6 |
| Grated cheddar cheese | 55.5 |
| Plain yoghurt | 26 |
| Lemon juice | 1.9 |
| Soy sauce | 2.2 |
| Calcium citrate malate (finely ground) | 6.2 |
| Minced garlic | 1.6 |

In particular, approximately 200 grams of the spread is prepared by mixing all ingredients in a food processor and blending until smooth.

### Example 11

Peanut butter balls are prepared by combining the following components:

| Component | Wt % |
|---|---|
| Creamy Peanut Butter | 31.69 |
| Margarine (softened) | 16.36 |
| Powdered Sugar | 46 |
| Glucosamine HCl | 2.76 |
| Calcium Carbonate | 1.39 |
| Citric Acid | 0.88 |
| Malic Acid | 0.92 |

The components are mixed together and formed into 1 inch balls. Chill. Melt chocolate chips and dip the peanut butter balls in the melted chocolate.

### Example 12

A peanut butter is formed by combining the following components:

| Component | Wt % |
|---|---|
| Creamy Peanut Butter | 91.69 |
| Glucosamine HCl | 4.3 |
| Calcium citrate malate (milled) | 4.01 |

### Example 13

Yogurt is formed by combining the following components. The final pH of the yogurt is about 4.4.

| Component | Wt % |
|---|---|
| Yogurt | 98.74 |
| Glucosamine HCl | 0.65 |
| Calcium citrate malate (milled) | 0.61 |

### Example 14

A glaze is formed by combining the following components:

| Component | Wt % |
|---|---|
| Water | 13.2 |
| Glucosamine HCl | 0.57 |
| Malic Acid | 0.19 |
| Citric Acid | 0.19 |
| Calcium Carbonate | 0.28 |
| Powdered Sugar | 85.58 |

### Example 15

Lemon bars are prepared by combining the following components as indicated:

| Component | Wt % |
|---|---|
| Flour | 22.1 |
| Calcium carbonate | 0.5 |
| Citric acid | 0.3 |
| Malic acid | 0.3 |
| Powdered sugar | 7.4 |
| Butter | 16 |
| Sugar | 27.7 |
| Baking powder | 0.2 |
| Glucosamine HCl | 1.0 |
| Lemon juice | 8.5 |
| Eggs | 16 |

Mix flour and about half of the powdered sugar together. Blend in butter. Press this mixture into pan for crust. Bake for 30 minutes at 350° F. Beat flour, sugar, baking powder, lemon juice and eggs together. Pour this mixture over the baked crust and bake again at 350° F. for another 25 minutes. Dust the top with the remaining powdered sugar, cool and serve.

### Example 16

High quality caramel chews are prepared by combining the following components as indicated:

| Component | Grams |
|---|---|
| Corn syrup | 375 |
| Granulated sugar | 300 |
| Invert sugar | 75 |
| Dairy Cream (30%) | 60 |
| Hydrogenated vegetable oil | 30 |
| Unsweetened evaporated milk | 480 |
| Calcium citrate malate (milled) | 70 |
| Glucosamine hydrochloride | 75 |
| Dairy buffer | 15 |
| Salt | to taste |

Combine corn syrup, sugar, invert sugar, dairy cream and vegetable oil into a pan and mix. Heat until temperature reaches 240° F. Add evaporated milk gradually with constant stirring until it boils. Continue to heat to 244° F. Add butter. Salt may be added to taste. Pour into non-stick pan, cool, and cut into chews.

### Example 17

Hard lemon candies are prepared by combining the following components as indicated:

| Component | Grams |
|---|---|
| Sugar | 200 |
| Light corn syrup | 63 |
| Water | 60 |
| Lemon flavor | 0.5 |
| Glucosamine hydrochloride | 16 |
| Calcium citrate malate | 14.9 |

Cook sugar, corn syrup, and water (disperse glucosamine and calcium citrate malate in water) in a double boiler until a temperature of 270° F. is reached on a candy thermometer. Remove from heat and stir in flavor. Pour into pan or molds.

### Example 18

Two separate dry compositions suitable for dilution to provide a ready-to-drink composition are prepared having the following components. These dry compositions are packaged in single-serving pouches, which are transportable and convenient for use by the consumer.

| Component | Wt % | Wt % |
|---|---|---|
| Glucosamine HCl | 5.87 | 6.81 |
| Citric acid | 1.67 | 1.94 |
| Malic acid | 1.75 | 2.03 |
| Calcium hydroxide | 1.94 | 2.24 |
| Ascorbic acid | 0.27 | 0.31 |
| Color | 0.067 | 0.077 |
| Flavor | 2.35 | 2.72 |
| Xanthan gum | 0.16 | 0.19 |
| Citric acid (beyond that which | 3.29 | 3.81 |
| is used for second component) | | |
| Fructose | 0 | quantum satis |
| Sucrose | quantum satis | 0 |

The components for each composition are blended to form a powdered mixture. Approximately 30 grams of each composition may be diluted with water to provide a 250 gram ready-to-drink beverage composition.

### Example 19

A low-calorie dry composition suitable for dilution to provide a ready-to-drink composition is prepared having the following components. The dry composition is packaged in a single-serving pouch, which is transportable and convenient for use by the consumer.

| Component | Wt % |
|---|---|
| Glucosamine HCl | 17.75 |
| Citric acid | 5.04 |
| Malic acid | 5.29 |
| Calcium hydroxide | 5.85 |
| Ascorbic acid | 0.81 |
| Color | 0.2 |
| Flavor | 7.1 |
| Xanthan gum | 0.49 |
| Citric acid (beyond that which is used for | 9.94 |
| second component) | |
| Crystalline sucralose | 0.18 |
| Erythritol | 47.34 |

The components are blended to form a powdered mixture. Approximately 10 grams of the powdered mixture may be diluted with water to provide a 250 gram ready-to-drink beverage composition.

### Example 20

A 237 milliliter low-calorie beverage composition is prepared by combining the following components in a conventional manner:

| Component | Wt % |
|---|---|
| Ascorbic acid | 0.07 |
| Calcium disodium EDTA | 0.003 |
| Calcium hydroxide | 0.25 |
| Citric acid | 0.63 |
| Erythritol | 2.0 |
| Crystalline fructose | 2.0 |
| Glucosamine HCl | 0.75 |
| Malic acid | 0.22 |
| Sodium benzoate | 0.002 |
| Sodium carboxymethylcellulose | 0.03 |
| Sucralose (25%) | 0.03 |
| Xanthan gum | 0.006 |
| Juice concentrates | 2.0 |
| Colors | 0.007 |
| Flavor oils | 0.04 |
| Water | quantum satis |

Various flavors of the beverage composition may be formulated according to standard techniques, for example, orange, grapefruit and/or cranberry flavors.

What is claimed is:

1. A composition comprising:
(a) a first component selected from the group consisting of gelatin, cartilage, aminosugars, glycosaminoglycans, methylsulfonylmethane, precursors of methylsulfonylmethane, S-adenosylmethionine, salts thereof, and mixtures thereof;
(b) a second component comprising:
(i) a cation source comprising an element selected from the group consisting of calcium, potassium, magnesium, and mixtures thereof; and
(ii) an edible acid source.

2. A composition according to claim 1 wherein the cation source comprises calcium.

3. A composition according to claim 2 wherein the edible acid source is selected from the group consisting of lactic acid, citric acid, malic acid, fumaric acid, adipic acid, phosphoric acid, gluconic acid, tartaric acid, ascorbic acid, acetic acid, phosphoric acid, succinic acid, and mixtures thereof.

4. A composition according to claim 3 wherein:
(a) the aminosugars are selected from the group consisting of glucosamine, galactosamine, salts thereof and mixtures thereof; and
(b) the glycosaminoglycans are selected from the group consisting of chondroitin, hyaluronic acid, keratan, heparin, dermatin, salts thereof, and mixtures thereof.

5. A composition according to claim 4 wherein:
(a) the aminosugars are selected from the group consisting of glucosamine and salts thereof, and
(b) the glycosaminoglycans are selected from the group consisting of chondroitin and salts thereof.

6. A composition according to claim 5 wherein the edible acid source is selected from the group consisting of citric acid, malic acid, and mixtures thereof.

7. A composition according to claim 6 wherein the second component is calcium citrate malate.

8. A composition according to claim 7 wherein the first component is selected from the group consisting of glucosamine sulfate, glucosamine potassium sulfate, glucosamine hydrochloride, and N-acetyl glucosamine.

9. A composition according to claim 7 wherein the first component is selected from the group consisting of chondroitin and salts thereof.

10. A composition according to claim 7 wherein the first component is a mixture of:
(a) one or more aminosugars; and
(b) one or more glycosaminoglycans.

11. A composition according to claim 10 wherein the first component is a mixture of:
(a) one or more aminosugars, wherein the aminosugars are selected from the group consisting of glucosamine sulfate, glucosamine potassium sulfate, and glucosamine hydrochloride; and
(b) one or more glycosaminoglycans.

12. A composition according to claim 11 wherein the glycosaminoglycan is chondroitin sulfate.

13. A composition according to claim 11 wherein the glycosaminoglycan is hyaluronic acid.

14. A composition according to claim 1 further comprising one or more omega-3-fatty acids.

15. A composition according to claim 7 further comprising one or more omega-3-fatty acids.

16. A composition according to claim 15 wherein the omega-3-fatty acid is selected from the group consisting of eicosapentaenoic acid, docosahexaenoic acid, and mixtures thereof.

17. A beverage comprising:
(a) a composition according to claim 1; and
(b) one or more beverage components selected from the group consisting of water, fruit juice, tea solids, milk solids, fruit flavors, botanical flavors, and mixtures thereof.

18. A beverage comprising:
(a) a composition according to claim 7; and
(b) one or more beverage components selected from the group consisting of water, fruit juice, tea solids, milk solids, fruit flavors, botanical flavors, and mixtures thereof.

19. A beverage according to claim 18 further comprising one or more omega-3-fatty acids.

20. A beverage according to claim 18 further comprising one or more nutrients additional to the second component.

21. A beverage comprising:
(a) a composition according to claim 8; and
(b) one or more beverage components selected from the group consisting of water, fruit juice, tea solids, milk solids, fruit flavors, botanical flavors, and mixtures thereof.

22. A kit comprising:
(a) a composition according to claim 1; and
(b) information that use of the composition promotes a benefit selected from the group consisting ofjoint health, bone health, cardiac health, and anti-inflammation.

23. A kit comprising:
(a) a composition according to claim 7; and
(b) information that use of the composition promotes a benefit selected from the group consisting ofjoint health, bone health, cardiac health, and anti-inflammation.

24. A kit according to claim 23 wherein the composition is a beverage.

25. A kit comprising:
(a) a composition according to claim 8; and
(b) information that use of the composition promotes a benefit selected from the group consisting of joint health, bone health, cardiac health, and anti-inflammation.

26. A kit according to claim 25 wherein the composition is a beverage.

27. A method of treating joint function, bone function, cardiac function, or inflammation comprising administering to a mammal a composition according to claim 1.

28. A method according to claim 27 wherein the administration is oral and the composition is a beverage.

29. A method of treating joint function, bone function, cardiac function, or inflammation comprising administering to a mammal a composition according to claim 7.

30. A method according to claim 29 wherein the administration is oral and the composition is a beverage.

31. A method of treating joint function, bone function, cardiac function, or inflammation comprising administering to a mammal a composition according to claim 8.

32. A method according to claim 31 wherein the administration is oral and the composition is a beverage.
